(19) United States
(12) Patent Application Publication
(10) Pub. No.: US 2025/0209690 A1
(43) Pub. Date: Jun. 26, 2025

(54) RECONSTRUCTION OF A TRUNCATED MEDICAL IMAGE

(71) Applicant: Novocure GmbH, Robot D4 (CH)

(72) Inventors: Oren PELES ZEEVI, Haifa (IL); Amit FELDMAN, Haifa (IL)

(73) Assignee: Nococure GmbH, Robot D4 (CH)

(21) Appl. No.: 18/968,023

(22) Filed: Dec. 4, 2024

Related U.S. Application Data
(60) Provisional application No. 63/613,796, filed on Dec. 22, 2023.

(51) Int. Cl. G06T 11/00; A61N 1/08; G06T 7/62; G06T 17/00; G16H 20/40; G16H 30/40

(52) U.S. Cl. CPC G06T 11/006; A61N 1/08; G06T 7/62; G06T 11/005; G06T 17/00; G16H 20/40; G16H 30/40; G06T 2207/10081; G06T 2207/10088; G06T 2207/10104; G06T 2207/20081; G06T 2207/20084; G06T 2207/20092; G06T 2207/30061; G06T 2210/41; G06T 2211/432

(57) ABSTRACT

A method for reconstructing a truncated medical image includes: accessing from memory a medical image of a subject, the medical image comprising voxels; determining the medical image has a truncated portion of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values.

### CROSS-REFERENCE TO RELATED APPLICATIONS

This application claims priority to U.S. Provisional Patent Application No. 63/613,796, filed Dec. 22, 2023, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 is a flowchart depicting an example method for training a machine learning model to reconstruct a truncated medical image.

FIGS. 2A and 2B (collectively referred to as FIG. 2) are a flowchart depicting an example method for reconstructing a truncated medical image and, using the reconstructed medical image, selecting transducers for delivering alternating electric fields to a subject.

FIG. 3 is a flowchart depicting an example method for determining whether a medical image has a truncated portion of the subject.

FIGS. 4A and 4B are flowcharts each depicting an example method for determining whether a medical image has a truncated portion of the subject.

FIG. 5 is an example medical image having a truncated portion.

FIGS. 6A, 6B, and 6C depict an example of a medical image processed according to an example embodiment.

FIGS. 6D, 6E, and 6F depict an example of a medical image processed according to an example embodiment.

FIG. 7 depicts an example system to apply alternating electric fields to a subject.

FIG. 8 depicts an example placement of transducers on a subject's head.

FIG. 9 depicts an example computer apparatus for use with the embodiments herein.

Various embodiments are described in detail below with reference to the accompanying drawings, where like reference numerals represent like elements.

### DESCRIPTION OF EMBODIMENTS

This application describes exemplary techniques to computationally reconstruct a truncated medical image and further provide one or more recommended transducer layouts for applying TTFields to a subject based on the reconstructed medical image. As an example, a truncated medical image may result from generating a medical image of a torso of a subject, and the left and right sides of the subject may be truncated in the medical image.

Previously, a truncated medical image may have been acceptable when considering a radiation treatment plan to treat a tumor of a subject. However, a truncated medical image may be unacceptable for TTFields treatment planning, which may require knowing conductivities of certain areas of the subject to determine energy flow for TTFields treatment planning. If a portion of the subject is missing due to truncation in the medical image (i.e., a truncated medical image), conductivities of this missing portion of the subject will not be known, and, as such, energy flow in this missing portion cannot be determined, which results in the inability to provide accurate TTFields treatment planning. If the subject only has one medical image, which is a truncated medical image, the subject would need to re-schedule to obtain a medical image without truncation, thereby delaying TTFields treatment as well as increasing the costs of the TTFields treatment. If the subject has multiple medical images including a truncated medical image having particularly relevant information, the truncated medical image may not be used for TTFields treatment planning, thereby losing the particularly relevant information.

To alleviate these problems, the inventors discovered computational techniques to reconstruct a truncated medical image based on a trained machine learning model to obtain a reconstructed medical image, which may then be used for TTFields treatment planning. As such, medical images with truncated portions that may have previously not been usable for TTFields treatment planning may now be used for TTFields treatment planning by employing the inventive techniques. The inventive techniques are particularly integrated into a practical application of providing a reconstructed medical image, which may be used in further computational processing techniques, such as generating one or more recommended transducer layouts for applying TTFields to the subject based on the reconstructed medical image. With the inventive techniques, the process of generating a reconstructed medical image may result in quicker, less expensive, and/or more accurate TTFields treatment planning.

In some embodiments, the inventive techniques include training a machine learning model using a plurality of existing medical images of a plurality of the subjects to obtain a trained machine learning model that produces a reconstructed medical image from a truncated medical image. With the reconstructed medical image generated with the inventive techniques, a plurality of transducer layouts for application of TTFields to the subject may be generated, and one or more of the transducer layouts may be recommended for applying TTFields treatment to the subject.

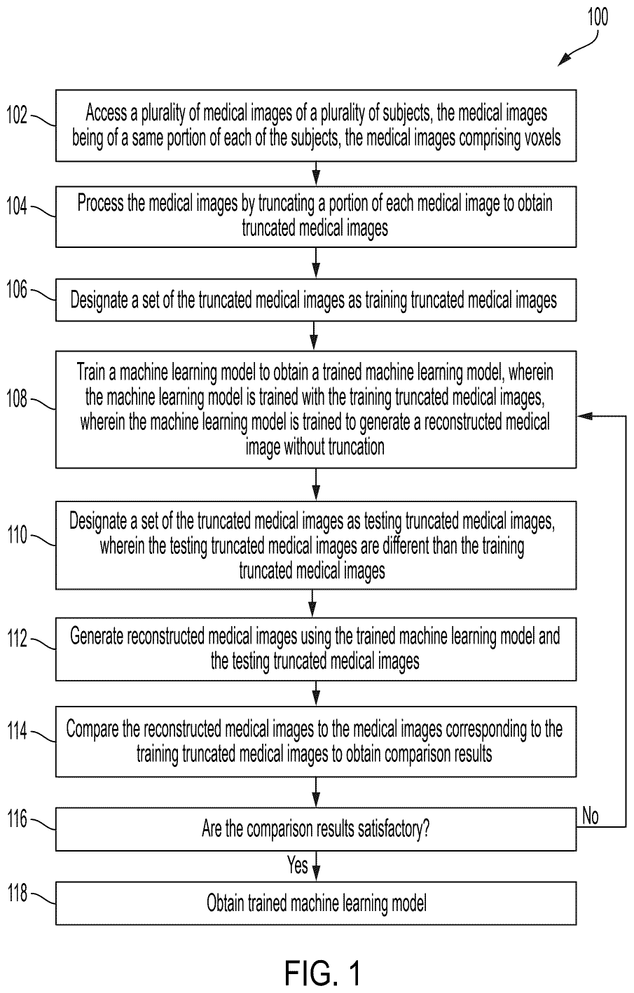

FIG. 1 depicts an example method 100 for training a machine learning model to reconstruct a truncated medical image. Certain steps of the method 100 are described as computer-implemented steps. The computer may include one or more processors and memory accessible by the one or more processors, the memory storing instructions that when executed by the one or more processors cause the computer to perform the relevant steps of the method 100. Modifications, additions, or omissions may be made to method 100. While an order of operations is indicated in FIG. 1 for illustrative purposes, the timing and ordering of such operations may vary where appropriate without negating the purpose and advantages of the examples set forth in detail herein.

With reference to FIG. 1, at step 102, the method 100 may include accessing a plurality of medical images of a plurality of subjects. In some embodiments, the medical image may be stored at and accessible from a computer memory locally or over a network. The plurality of medical images may include at least one of a magnetic resonance imaging (MRI) medical image, a computed tomography (CT) medical image, or a positron emission tomography (PET) medical image. A medical image may include a plurality of voxels and a plurality of slices, where each slice has a plurality of voxels. The medical images may be of a same portion of each of the subjects. In some embodiments, the same portion of each of the subjects in the plurality of medical images may be of a torso and/or a head of the corresponding subject. As one example, the plurality of medical images may be of a torso of each of the plurality of subjects. In some embodiments, step 102 may be computer implemented.

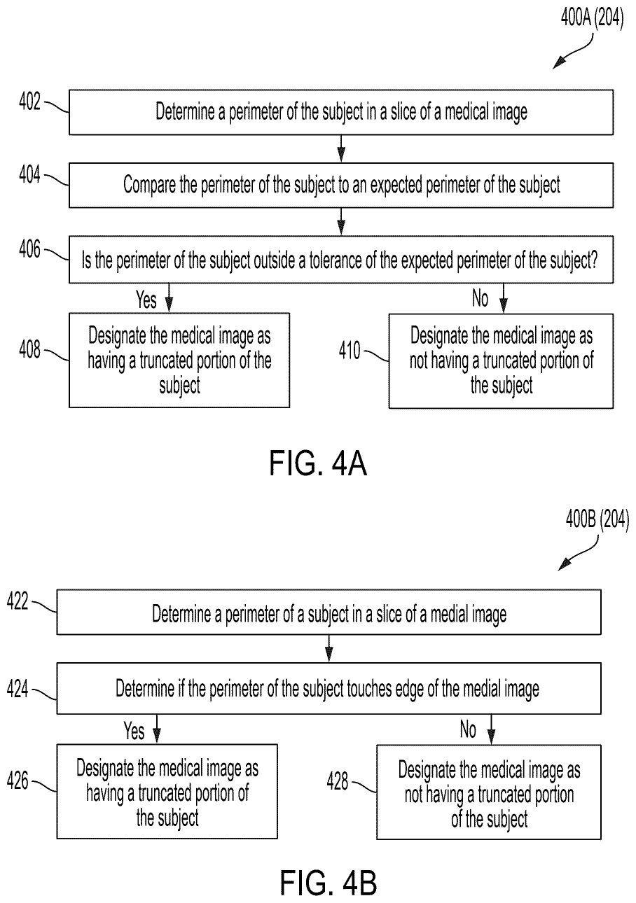

At step 104, the method 100 may include processing the medical images by truncating a portion of each medical image to obtain truncated medical images. As an example, the truncated portion of each medical image may correspond to a right side and a left side of each subject. FIG. 5 shows an example medical image having a truncated portion including a right side 504 and a left side 502. Other portions of each subject may be truncated. The truncated portion of the subject may include voxels having non-tissue image values. In some embodiments, the non-tissue image values of the truncated portion may be identical image values. In some embodiments, the non-tissue image values of the truncated portion may correspond to background image values. In some embodiments, step 104 may be computer implemented. In some embodiments, step 104 may be implemented based on user input, such as designating portions of each medical image to be truncated, or such as designating portions of one medical image, or a few medical images, to be truncated, where the computer performs the truncation of the medical images based thereon.

At step 106, the method 100 may include designating a set of the truncated medical images as training truncated medical images. In some embodiments, step 106 may be computer implemented. In some embodiments, step 106 may be implemented based on user input, such as identifying which and/or how many of the truncated medical images should be designated as the training truncated medical images.

At step 108, the method 100 may include training a machine learning model to obtain a trained machine learning model, where the machine learning model is trained with the training truncated medical images, and where the machine learning model is trained to generate a reconstructed medical image without truncation. In some embodiments, step 108 may be computer implemented. Prior to providing the training truncated medical images to the machine learning model, a quality test may be performed on the training truncated medical images by, for example, human eye or calculating a loss value of a network itself, to help provide better training quality.

The machine learning model may include one or more algorithms and/or structures. In some embodiments, the machine learning model may be an unsupervised machine learning model. In some embodiments, the trained machine learning model may be a deep learning neural network. In some embodiments, the machine learning model may include at least one of a generative adversarial network (GAN), a MedGAN, a super resolution GAN, a pix2pix GAN, a cycleGAN, a discoGAN, a fila-sGAN, a projective adversarial network (PAN), a variational autoencoder (VAE), or an unsupervised neural network. As one example, the machine learning model may be a GAN including a deconvolutional neural network as a generator and a convolutional neural network as a discriminator.

At step 110, the method 100 may include designating a set of the truncated medical images as testing truncated medical images. The testing truncated medical images may be different than the training truncated medical images. In some embodiments, each truncated medical images may be identified as either a training truncated medical image in step 106 or a testing truncated medical image in step 110. In some embodiments, step 110 may be computer implemented. In some embodiments, step 110 may be implemented based on user input, such as identifying which and/or how many of the truncated medical images should be designated as the testing truncated medical images. A quality test applied may also be applied to the testing truncated medical images.

At step 112, the method 100 may include generating reconstructed medical images using the trained machine learning model and the testing truncated medical images. In some embodiments, step 112 may be computer implemented.

At step 114, the method 100 may include comparing the reconstructed medical images to the medical images corresponding to the training truncated medical images to obtain comparison results. In some embodiments, step 114 may be computer implemented.

At step 116, the method 100 may include determining if the comparison results are satisfactory or unsatisfactory. In some embodiments, the comparison may be determined as satisfactory if the reconstructed medical images and the medical images corresponding to the training truncated medical images have a same dose of TTFields treatment and/or a same ranking of transducer layouts for administration of TTFields. Upon determining that that the comparison results are satisfactory, the process goes to step 118.

Upon determining that the comparison results are unsatisfactory, the process goes back to repeat steps 108-114 to further train the machine learning model and to obtain a re-trained machine learning model. In particular, this re-training may involve utilizing updated training truncated medical images. In some embodiments, step 116 may be computer implemented.

At step 118, the method 100 may include obtaining the trained machine learning model. The trained machine learning model may be obtained once the comparison results are determined to be satisfactory. In some embodiments, step 118 may be computer implemented.

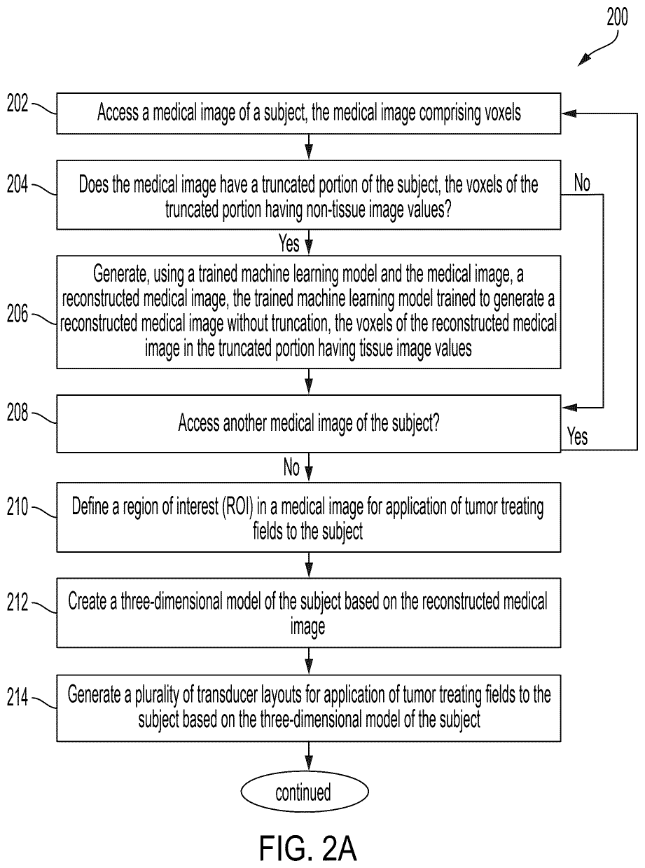

FIGS. 2A and 2B depict an example method 200 for reconstructing a truncated medical image and, using the reconstructed medical image, selecting transducers for delivering alternating electric fields to a subject. Certain steps of the method 200 are described as computer-implemented steps. The computer may include one or more processors and memory accessible by the one or more processors, the memory storing instructions that when executed by the one or more processors cause the computer to perform the relevant steps of the method 200. Modifications, additions, or omissions may be made to method 100. While an order of operations is indicated in FIG. 2 for illustrative purposes, the timing and ordering of such operations may vary where appropriate without negating the purpose and advantages of the examples set forth in detail herein.

With reference to FIG. 2A, at step 202, the method 200 may include accessing a medical image of a subject, where the medical image comprising voxels. In some embodiments, the medical image may be stored at and accessible from a computer memory locally or over a network. In some embodiments, the medical image of the subject may include an image of torso and/or a head of the subject. As one example, the medical image may include a lung of the subject. In some embodiments, step 202 may be computer implemented.

At step 204, the method 200 may include determining if the medical image has a truncated portion of the subject. FIG. 5 shows an example medical image having a truncated portion. The truncated portion of the subject may include voxels having non-tissue image values. In some embodiments, the non-tissue image values of the truncated portion may be identical image values. In some embodiments, the non-tissue image values of the truncated portion may correspond to background image values.

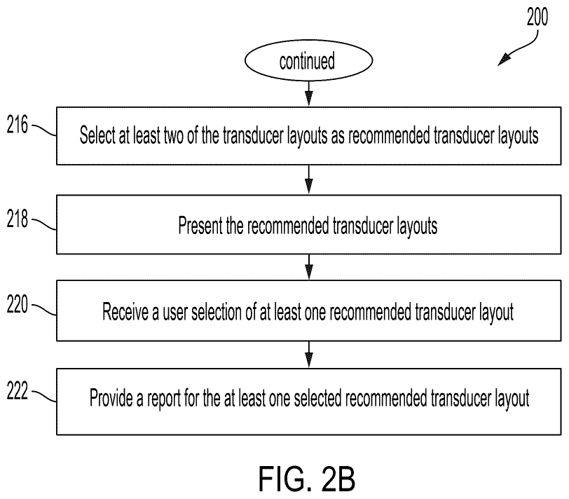

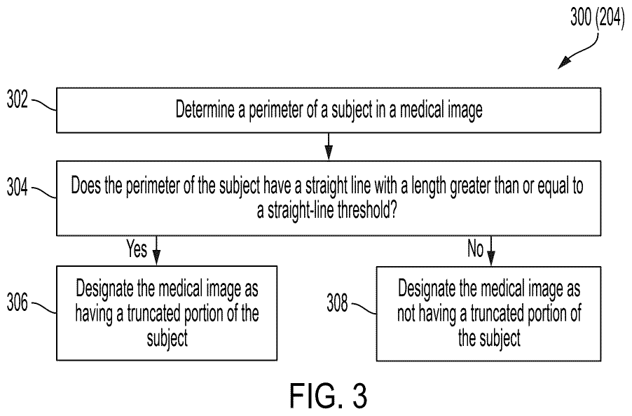

In some embodiments, step 204 may be computer implemented. In some embodiments, the medical image may be determined as having a truncated portion of the subject upon determining that the subject depicted in the medical image has at least two flat surfaces. In some embodiments, the medical image may be determined as having a truncated portion of the subject upon determining that the subject depicted in the medical image has at least one flat surface. In some embodiments, the determination may be based on a determined perimeter of the subject in the medical image. In some embodiments, step 204 may be manually performed by a user or based on input from a user. As an example, the determination may include presenting the medical image on a display and receiving a user input identifying the medical image as having a truncated portion of the subject. Additional details regard step 204 are illustrated in FIG. 3 and FIGS. 4A and 4B and the accompanying descriptions. Upon determining that the medical image has a truncated portion of the subject, the process goes to step 206. Otherwise, the process goes to step 208.

At step 206, the method 200 may include generating, using a trained machine learning model and the medical image, a reconstructed medical image. The voxels of the reconstructed medical image in the truncated portion having tissue image values. The trained machine learning model is trained to generate a reconstructed medical image without truncation. The trained machine learning model may be obtained according to method 100 of FIG. 1.

At step 208, the method 200 may include determining if there is another medical image of the subject to access. Upon determining yes, the process goes back to repeat steps 202-206. Upon determining no, the process continues to step 210.

At step 210, the method 200 may include defining a region of interest (ROI) in the medical images (in one or more reconstructed medical images and/or one or more medical images that did not need to be reconstructed) for application of TTFields to the subject. The ROI may define where the TTFields are to focus. In some embodiments, the ROI may be defined based on user input or may be defined partially or entirely by the computer.

At step 212, the method 200 may include creating a three-dimensional (3D) model of the subject based on the reconstructed medical image(s) and the medical images that did not need to be reconstructed, where the 3D model of the subject may include the ROI. In some embodiments, the 3D model may include a 3D conductivity map depicting electrical conductivity of tissues of the subject. In some embodiments, the 3D model may be generated by the computer and may be based on user input

In some embodiments, creating the 3D model may include performing calculations to determine conductivity of the tissues of the subject based on the reconstructed medical image(s) and any other medical images, and the tissue types therein. As one example, creating the 3D model may include assigning tissue types and associated conductivities to voxels of the 3D model of the subject. In some embodiments, creating the 3D model of the subject may include automatically segmenting normal tissue in the medical images. In some embodiments, the 3D model may be created based on user input, such as user approval on a 3D conductivity map associated with the 3D model.

At step 214, the method 200 may include generating a plurality of transducer layouts for application of TTFields to the subject based on the 3D model of the subject, which is created based on the reconstructed medical image at step 212. The transducer layouts may define one or more locations, relative to the subject, for placing one or more transducers. In some embodiments, a transducer layout may include four locations on the subject to place four respective transducers, such as on a head or torso of the subject. In some embodiments, the plurality of the transducer layouts may include two locations on the subject to place two respective transducers, such as on a head or torso of the subject. In some embodiments, the transducer may include one electrode element or a plurality of electrode elements. The electrode elements may be any suitable type or material. For example, at least one electrode element may include a ceramic dielectric layer, a polymer film, and/or the like including combinations and/or multiples thereof. In some embodiments, step 214 may be computer implemented.

At step 216, the method 200 may include selecting one or more of the transducer layouts as recommended transducer layouts. In some embodiments, one, two, three, four or more transducer layouts may be selected as recommended transducer layouts. In some embodiments, step 216 may be computer implemented. In some embodiments, the selection may be based on user input.

At step 218, the method 200 may include presenting the recommended transducer layouts on a display. In some embodiments, the recommended transducer layouts may be presented on a display through one or more output devices for a user's consideration.

At step 220, the method 200 may include receiving a user selection of at least one recommended transducer layout. The user selection may indicate a preferred transducer layout to apply the TTFields in the ROI of the subject.

At step 222, the method 200 may include providing a report for the at least one selected recommended transducer layout, where the report indicates the selection result from step 122.

FIG. 3 depicts an example method 300 for determining whether a medical image has a truncated portion of the subject. The method 300 may implement step 204 of FIG. 2A. The method 300 may be implemented by a computer, the computer including one or more processors and memory accessible by the one or more processors, the memory storing instructions that when executed by the one or more processors cause the computer to perform the relevant steps of the method 300. Modifications, additions, or omissions may be made to method 300. While an order of operations is indicated in FIG. 3 for illustrative purposes, the timing and ordering of such operations may vary where appropriate without negating the purpose and advantages of the examples set forth in detail herein.

With reference to FIG. 3, at step 302, the method 300 may include determining a perimeter of a subject in a medical image. The perimeter may be determined using automatic edge detection by identifying voxels with background image values compared to voxels with non-background image values.

At step 304, the method 300 may include determining if the perimeter of the subject has a straight line with a length greater than or equal to a straight-line threshold. In some embodiments, the straight-line threshold may be a maximum straight-line length allowed in a medical image so as to be considered not truncated. In some embodiments, the straight-line threshold may be determined by a user. As one example, the straight-line threshold may be between 3 cm to 5 cm. Upon determining that the perimeter of the subject has a straight line with a length greater than or equal to the straight-line threshold in step 304, the process goes to step 306. Upon determining that the perimeter of the subject does not have a straight line with a length greater than or equal to the straight-line threshold in step 304, the process goes to step 308.

In step 306, the medical image is designated as having a truncated portion of the subject and, hence, is designated as a truncated medical image.

In step 308, the medical image is designated as not having a truncated portion of the subject.

As an example of FIG. 3, if the perimeter of the subject has a straight line equal to or greater than 5 cm in step 304, the medical image is designated in step 306 as having a truncated portion of the subject. Otherwise, the medical image is designated in step 308 as not having a truncated portion of the subject.

FIGS. 4A and 4B depict example methods 400A and 400B for determining whether a medical image has a truncated portion of the subject. The methods 400A and 400B may implement step 204 of FIG. 2A. The methods 400A and 400B may be implemented by a computer, the computer including one or more processors and memory accessible by the one or more processors, the memory storing instructions that when executed by the one or more processors cause the computer to perform the relevant steps of the methods 400A and 400B. Modifications, additions, or omissions may be made to methods 400A and 400B. While an order of operations is indicated in FIGS. 4A and 4B for illustrative purposes, the timing and ordering of such operations may vary where appropriate without negating the purpose and advantages of the examples set forth in detail herein.

With reference to FIG. 4A, at step 402, the method 400A may include determining a perimeter of a subject in a slice of a medical image. The perimeter may be determined using automatic edge detection by identifying voxels with background image values compared to voxels with non-background image values.

At step 404, the method 400A may include comparing the perimeter of the subject to an expected perimeter of the subject. In some embodiments, the expected perimeter of the subject may be based on a view of the subject in the slice of the medical image and at least one of gender, age, body mass index, a perimeter measurement, a height measurement, or a measurement of a portion of the subject.

At step 406, the method 400A may include determining if the perimeter of the subject is outside a tolerance of the expected perimeter of the subject. The tolerance of the expected perimeter of the subject may be represented by a percentage. In some embodiments, the tolerance of the expect perimeter of the subject may be determined by the user. As one example, the tolerance of the expected perimeter of the subject may between 3% and 5%. Upon determining that the perimeter of the subject is outside a tolerance of the expected perimeter of the subject, the process goes to step 408. Upon determining that the perimeter of the subject is not outside a tolerance of the expected perimeter of the subject, the process goes to step 410,

In step 408, the medical image is designated as having a truncated portion of the subject and, hence, is designated as a truncated medical image.

In step 410, the medical image is designated as not having a truncated portion of the subject.

As an example of FIG. 4A, assuming that the expected perimeter of the subject is 100 cm and assuming the tolerance of the expected perimeter of the subject is 5%, the medical image may be designated as having a truncated portion of the subject if the perimeter of the subject is less than 95 cm or greater than 105 cm. The medical image may be designated as not having a truncated portion of the subject if the perimeter of the subject falls between 95 cm and 105 cm.

Turning to FIG. 4B, at step 422, the method 400B may include determining a perimeter of a subject in a slice of a medical image. The perimeter may be determined using automatic edge detection by identifying voxels with background image values compared to voxels with non-background image values.

At step 424, the method 400B may include determining if the perimeter of the subject touches one or more edges of the medical image. In some embodiments, the perimeter of the subject touches the one or more edges of the medical image if one or more straight lines are formed at the subject's area, rather than the one or more edges of the medical image merely being tangent to the perimeter of the subject. Upon determining that the perimeter of the subject touches one or more edges of the medical image, the process goes to step 426. Upon determining that the perimeter of the subject does not touch an edge of the medical image, the process goes to step 428.

In step 426, the medical image is designated as having a truncated portion of the subject and, hence, is designated as a truncated medical image.

In step 428, the medical image is designated as not having a truncated portion of the subject.

FIG. 5 shows an example medical image having a truncated portion. According to FIG. 5, the medical image has a truncated portion 502 and a truncated portion 504. Each of the truncated portions 502 and 504 has a straight line. In some embodiments, each of the straight lines may have a length greater than or equal to a straight-line threshold, as discussed in method 300. In some embodiments, due to the truncated portions 502 and 504, the perimeter of the subject would not be an exact match for a perimeter of a non-truncated subject, and may be outside a tolerance of an expected perimeter of the subject, as discussed in method 400A. In some embodiments, the perimeter of subject shown in FIG. 5 touches the left and right edges of the medical image at portions 502 and 504, respectively, as discussed in method 400B.

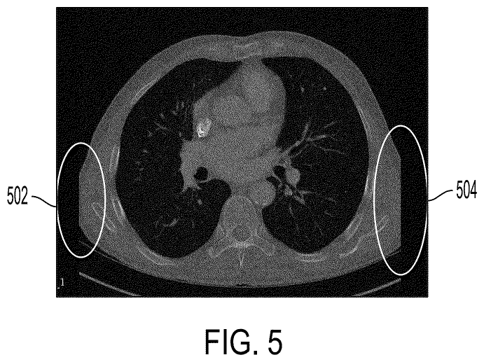

FIGS. 6A, 6B, and 6C depict examples of a first medical image processed according to an example embodiment. FIGS. 6A, 6B, and 6C depict a transverse plane view of a torso of a subject. FIG. 6A depicts a slice in the first medical image prior to processing. The first medical image of FIG. 6A is truncated, resulting in a truncated medical image in FIG. 6B. According to an example embodiment, the truncated medical image of FIG. 6B is reconstructed, resulting in a reconstructed medical image of FIG. 6C.

FIGS. 6D, 6E, and 6F depict examples of a second medical image processed according to an example embodiment. FIGS. 6D, 6E, and 6F depict a transverse plane view of a torso of a different subject from those in FIGS. 6A, 6B, and 6C. FIG. 6D depicts a slice in the second medical image prior to processing. The medical image of FIG. 6D is truncated, resulting in a truncated medical image of the subject in FIG. 6E. According to an example embodiment, the truncated medical image of FIG. 6E is reconstructed, resulting in a reconstructed medical image of FIG. 6F.

In comparing FIG. 6A to FIG. 6C and in comparing FIG. 6D to FIG. 6F, the reconstructed medical images in FIG. 6C and FIG. 6F are fair representations of the medical images in FIGS. 6A and 6D. As such, the reconstructed medical images in FIG. 6C and FIG. 6F are of sufficient quality to be used for TTFields treatment planning. However, the truncated medical images in FIG. 6B and FIG. 6E are of insufficient quality to be used for TTFields treatment planning, due to the truncations on the left and right sides of each image.

### Exemplary Apparatuses

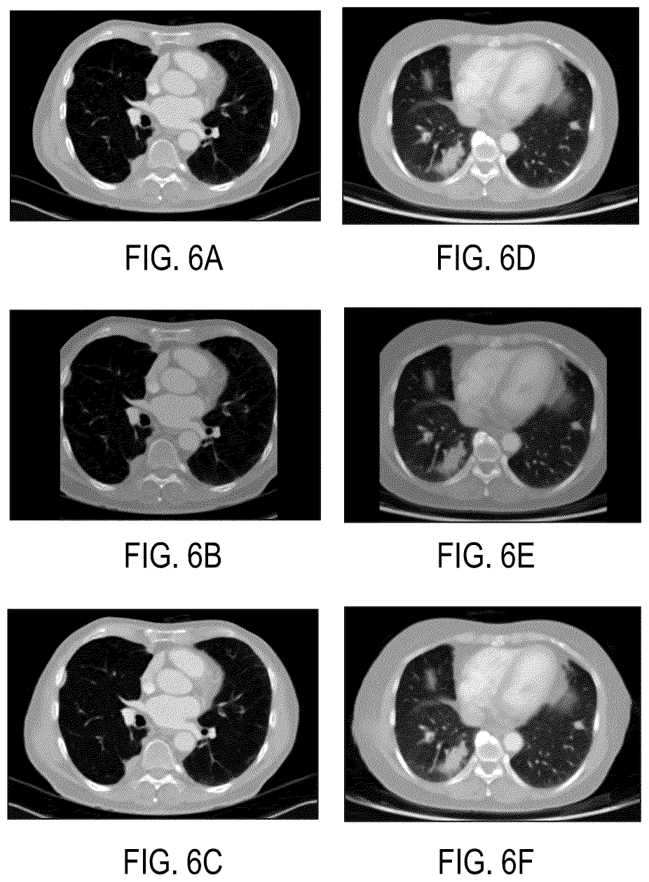

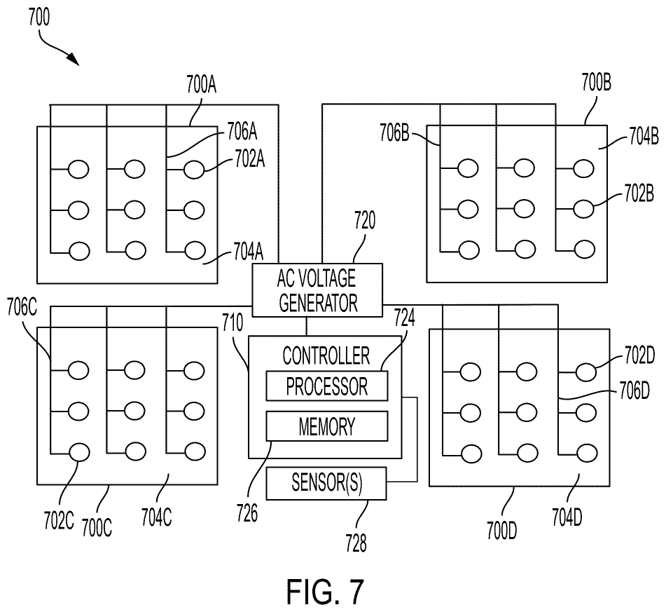

FIG. 7 depicts an example system 700 to apply alternating electric fields (e.g., TTFields) to a subject's body. The system may be used for treating a target region of a subject's body with an alternating electric field (e.g., TTFields). As an example, the target region may be in the subject's brain, and an alternating electric field may be delivered to the subject's body via two pairs of transducers positioned on a head of the subject's body (such as, for example, in FIG. 8, which has four transducers 800). As an example, the target region may be in the subject's torso, and an alternating electric field may be delivered to the subject's body via two pairs of transducers positioned on at least one of a thorax, an abdomen, or one or both thighs of the subject's body. As an example, a single pair of transducers may be used. Other transducer placements on the subject's body may be possible.

The example apparatus 700 depicts an example system having four transducers (or “transducer arrays”) 700A-D. Each transducer 700A-D may include substantially flat electrode elements 702A-D positioned on a substrate 704A-D and electrically and physically connected (e.g., through conductive wiring 706A-D). The substrates 704A-D may include, for example, cloth, foam, flexible plastic, and/or conductive medical gel. Two transducers (e.g., 700A and 700D) may be a first pair of transducers configured to apply an alternating electric field to a target region of the subject's body. The other two transducers (e.g., 700B and 700C) may be a second pair of transducers configured to similarly apply an alternating electric field to the target region.

The transducers 700A-D may be coupled to an AC voltage generator 720, and the system may further include a controller 710 communicatively coupled to the AC voltage generator 720. The controller 710 may include a computer having one or more processors 724 and memory 726 accessible by the one or more processors. The memory 726 may store instructions that when executed by the one or more processors control the AC voltage generator 720 to induce alternating electric fields between pairs of the transducers 700A-D according to one or more voltage waveforms and/or cause the computer to perform one or more methods disclosed herein. The controller 710 may monitor operations performed by the AC voltage generator 720 (e.g., via the processor(s) 724). One or more sensor(s) 728 may be coupled to the controller 710 for providing measurement values or other information to the controller 710.

In some embodiments, the voltage generation components may supply the transducers 700A-D with an electrical signal having an alternating current waveform at frequencies in a range from about 50 kHz to about 1 MHz and appropriate to deliver TTFields treatment to the subject's body

The electrode elements 702A-D may be capacitively coupled. As an example, the electrode elements 702A-D may be ceramic electrode elements coupled to each other via conductive wiring 706A-D. When viewed in a direction perpendicular to its face, the ceramic electrode elements may be circular shaped or non-circular shaped. In other embodiments, the array of electrode elements may not be capacitively coupled, and there is no dielectric material (such as ceramic, or high dielectric polymer layer) associated with the electrode elements.

The structure of the transducers 700A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one electrode element, or more than one electrode element). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

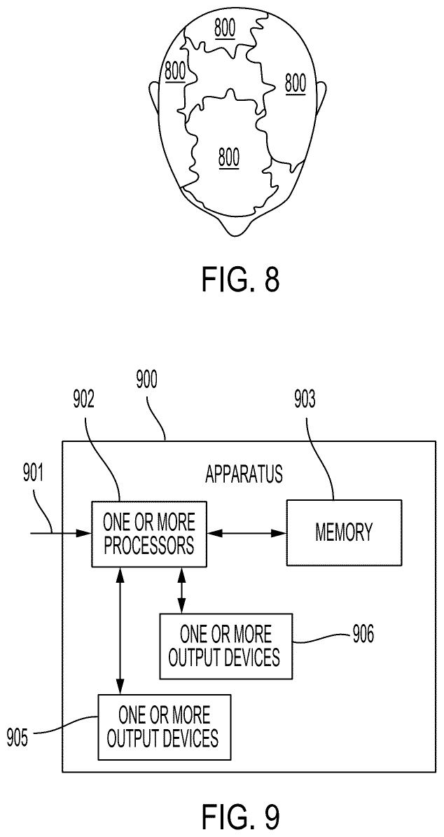

FIG. 9 depicts an example computer apparatus for use with one or more embodiments described herein. As an example, the apparatus 900 may be a computer to implement certain inventive techniques disclosed herein, such as reconstructing a truncated medical image and/or training a machine learning model to generate a reconstructed medical image. For example, steps 102 to 118 of FIG. 1 and steps 202 to 222 of FIGS. 2A and 2B may be performed by a computer, such as the apparatus 900. As an example, the apparatus 900 may be used as the controller 710 of FIG. 7, or as a separate computer apparatus located remote from the controller 710.

The apparatus 900 may include one or more processors 902, memory 903, one or more input devices 905, and one or more output devices 906.

Input to the apparatus 900 may be provided by one or more input devices 905, provided from one or more input devices in communication with the apparatus 900 via link 901 (e.g., a wired link or a wireless link; e.g., with a direct connection or over a network), and/or provided from another computer(s) in communication with the apparatus 900 via link 901. As an example, based on input 901, the one or more processor 902 may generate control signals to control the AC voltage generator 720. As an example, the input 701 may be user input. As an example, the input 701 may be from another computer in communication with the apparatus 700.

Output for the apparatus 900 may be provided by one or more output devices 906, provided to one or more output devices in communication with the apparatus 900 via link 901, and/or provided from another computer(s) in communication with the apparatus 900 via link 901. The one or more output devices 906 may provide the status of the operation of the invention, such as transducer layout selection, voltages being generated, and other operational information. The output device(s) 905 may provide visualization data according to certain embodiments of the invention.

In some embodiments, one or more input devices 905 and one or more output devices 906 may be combined into one or more unitary input/output devices (e.g., a touch screen).

In some embodiments, based on input from one or more input devices 905 or input from outside the apparatus 900 via the link 901, the one or more processors 902 may perform operations as described herein. As an example, user input may be received from the one or more input devices 905. As an example, input may be from another computer in communication with the apparatus 900 via link 901. As an example, input may be from one or more input devices in communication with the apparatus 900 via link 901.

In some embodiments, the one or more processors 902 may perform operations as described herein and provide results of the operations as output. As an example, output may be provided to the one or more output devices 906. As an example, output may be provided to another computer in communication with the apparatus 900 via link 901. As an example, output may be provided to one or more output devices in communication with the apparatus 900 via link 901.

The memory 903 may be accessible by the one or more processors 902 so that the one or more processors 902 may read information from and write information to the memory 903. The memory 903 may store instructions that, when executed by the one or more processors 902, implement one or more embodiments described herein. The memory 903 may be a non-transitory computer readable medium (or a non-transitory processor readable medium) containing a set of instructions thereon for reconstructing a truncated medical image and/or training a machine learning model to generate a reconstructed medical image, wherein when executed by a processor (such as one or more processors 902), the instructions cause the processor to perform one or more methods discussed herein.

The apparatus 900 may be an apparatus for reconstructing a truncated medical image and/or training a machine learning model to generate a reconstructed medical image, the apparatus including: one or more processors (such as one or more processors 902); and memory (such as memory 903) accessible by the one or more processors, the memory storing instructions that when executed by the one or more processors, cause the apparatus to perform one or more methods described herein.

The memory 903 may be a non-transitory processor readable medium containing a set of instructions thereon for reconstructing a truncated medical image and/or training a machine learning model to generate a reconstructed medical image, wherein when executed by one or more processors (such as one or more processors 902), the instructions cause the one or more processors to perform one or more methods described herein.

### Illustrative Embodiments

The invention includes other illustrative embodiments (“Embodiments”) as follows.

Embodiment 1. A computer-implemented method for reconstructing a truncated medical image, the method comprising: accessing from memory a medical image of a subject, the medical image comprising voxels; determining the medical image has a truncated portion of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values.

Embodiment 2: The method of Embodiment 1, wherein determining the medical image has a truncated portion of the subject comprises: determining the subject depicted in the medical image has at least two flat surfaces.

Embodiment 2A: The method of Embodiment 1, wherein determining the medical image has a truncated portion of the subject comprises: determining the subject depicted in the medical image has at least one flat surface.

Embodiment 3: The method of Embodiment 1, wherein determining the medical image has a truncated portion of the subject comprises: determining a perimeter of the subject in the medical image; determining if the perimeter of the subject has a straight line with a length greater than or equal to a straight-line threshold; if the perimeter of the subject has a straight line with a length greater than or equal to the straight-line threshold, designating the medical image as having a truncated portion of the subject; and if the perimeter of the subject does not have a straight line with a length greater than or equal to the straight-line threshold, designating the medical image as not having a truncated portion of the subject.

Embodiment 4: The method of Embodiment 1, wherein determining the medical image has a truncated portion of the subject comprises: determining a perimeter of the subject in a slice of the medical image; comparing the perimeter of the subject to an expected perimeter of the subject; if the perimeter of the subject is outside a tolerance of the expected perimeter of the subject, designating the medical image as having a truncated portion of the subject; and if the perimeter of the subject is not outside a tolerance of the expected perimeter of the subject, designating the medical image as not having a truncated portion of the subject.

Embodiment 5: The method of Embodiment 4, wherein the expected perimeter of the subject is based on a view of the subject in the slice of the medical image and at least one of gender, age, body mass index, a perimeter measurement, a height measurement, or a measurement of a portion of the subject.

Embodiment 6: The method of Embodiment 1, wherein determining the medical image has a truncated portion of the subject comprises: presenting the medical image on a display; and receiving a user input identifying the medical image as having a truncated portion of the subject.

Embodiment 7: The method of Embodiment 1, wherein the non-tissue image values of the truncated portion are identical image values.

Embodiment 8: The method of Embodiment 1, wherein the non-tissue image values of the truncated portion correspond to background image values.

Embodiment 9: The method of Embodiment 1, wherein the medical image comprises a torso of the subject.

Embodiment 10: The method of Embodiment 1, wherein the medical image comprises a lung of the subject.

Embodiment 11: The method of Embodiment 1, wherein the medical image comprises a computed tomography (CT) medical image, a magnetic resonance imaging (MRI) medical image, or a positron emission tomography (PET) medical image.

Embodiment 12: The method of Embodiment 1, further comprising: defining a region of interest (ROI) in at least one of the medical image or the reconstructed medical image for application of tumor treating fields to the subject; creating a three-dimensional model of the subject based on the reconstructed medical image, the three-dimensional model of the subject including the region of interest; generating a plurality of transducer layouts for application of tumor treating fields to the subject based on the three-dimensional model of the subject; selecting at least one of the transducer layouts as recommended transducer layouts; presenting the recommended transducer layouts; receiving a user selection of at least one recommended transducer layout; and providing a report for the at least one selected recommended transducer layout.

Embodiment 12A: A non-transitory processor readable medium containing a set of instructions thereon for reconstructing a truncated medical image, wherein when executed by a processor, the instructions cause the processor to perform a method comprising: accessing from memory a medical image of a subject, the medical image comprising voxels; determining the medical image has a truncated portion of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values.

Embodiment 12B: An apparatus for reconstructing a truncated medical image, the apparatus comprising: one or more processors; and memory accessible by the one or more processors, the memory storing instructions that when executed by the one or more processors, cause the apparatus to perform a method comprising: accessing from memory a medical image of a subject, the medical image comprising voxels; determining the medical image has a truncated portion of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values.

Embodiment 13: A computer-implemented method for reconstructing a truncated medical image, the method comprising: accessing from memory a plurality of medical images of a plurality of subjects, the medical images being of a torso of each of the subjects, the medical images comprising voxels; processing the medical images by truncating a portion of each medical image to obtain truncated medical images, wherein the truncated portion of each medical image corresponds to a right side and a left side of each subject; designating a set of the truncated medical images as training truncated medical images; and training a machine learning model to obtain a trained machine learning model, wherein the machine learning model is trained with the training truncated medical images, wherein the machine learning model is trained to generate a reconstructed medical image without truncation.

Embodiment 14: The method of Embodiment 13, further comprising: designating a set of the truncated medical images as testing truncated medical images, wherein the testing truncated medical images are different than the training truncated medical images; generating reconstructed medical images using the trained machine learning model and the testing truncated medical images; comparing the reconstructed medical images to the medical images corresponding to the training truncated medical images to obtain comparison results; and if the comparison results are unsatisfactory, re-training the trained machine learning model to obtain a re-trained machine learning model.

Embodiment 15: The method of Embodiment 13, wherein the machine learning model is a generative adversarial network.

Embodiment 16: The method of Embodiment 13, wherein the machine learning model is a generative adversarial network comprising a deconvolutional neural network as a generator and a convolutional neural network as a discriminator.

Embodiment 17: The method of Embodiment 13, wherein the trained machine learning model is a deep learning neural network.

Embodiment 18: The method of Embodiment 13, wherein the machine learning model is an unsupervised machine learning model

Embodiment 19: The method of Embodiment 13, wherein the machine learning model comprises a generative adversarial network (GAN), a MedGAN, a super resolution GAN, a pix2pix GAN, a cycleGAN, a discoGAN, a fila-sGAN, a projective adversarial network (PAN), a variational autoencoder (VAE), or an unsupervised neural network.

Embodiment 19A: A computer-implemented method for reconstructing a truncated medical image, the method comprising: accessing from memory a medical image of a subject, the medical image being of a torso of the subject, the medical image comprising voxels; determining the medical image has a truncated portion of the subject, the truncated portion of the medical image corresponding to a right side and a left side of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values; and generating a plurality of transducer layouts for application of tumor treating fields to the subject based on the reconstructed medical image.

Embodiment 19B: A non-transitory processor readable medium containing a set of instructions thereon for reconstructing a truncated medical image, wherein when executed by a processor, the instructions cause the processor to perform a method comprising: accessing from memory a medical image of a subject, the medical image being of a torso of the subject, the medical image comprising voxels; determining the medical image has a truncated portion of the subject, the truncated portion of the medical image corresponding to a right side and a left side of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values; and generating a plurality of transducer layouts for application of tumor treating fields to the subject based on the reconstructed medical image.

Embodiment 20: An apparatus for reconstructing a truncated medical image, the apparatus comprising: one or more processors; and memory accessible by the one or more processors, the memory storing instructions that when executed by the one or more processors, cause the apparatus to perform a method comprising: accessing from memory a medical image of a subject, the medical image being of a torso of the subject, the medical image comprising voxels; determining the medical image has a truncated portion of the subject, the truncated portion of the medical image corresponding to a right side and a left side of the subject, the voxels of the truncated portion having non-tissue image values; generating, using a trained machine learning model and the medical image, a reconstructed medical image, the trained machine learning model trained to generate a reconstructed medical image without truncation, the voxels of the reconstructed medical image in the truncated portion having tissue image values; and generating a plurality of transducer layouts for application of tumor treating fields to the subject based on the reconstructed medical image.

Embodiment 21: A method, machine, manufacture, and/or system substantially as shown and described.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A computer-implemented method for reconstructing a truncated medical image, the method comprising:
accessing from memory a medical image of a subject, the medical image comprising voxels;
determining the medical image has a truncated portion of the subject, the voxels of the truncated portion having non-tissue image values;
generating, using a trained machine learning model and the medical image, a reconstructed medical image, the trained machine learning model trained to generate a reconstructed medical image without truncation, the voxels of the reconstructed medical image in the truncated portion having tissue image values.

2. The method of claim 1, wherein determining the medical image has a truncated portion of the subject comprises:
determining the subject depicted in the medical image has at least two flat surfaces.

3. The method of claim 1, wherein determining the medical image has a truncated portion of the subject comprises:
determining a perimeter of the subject in the medical image;
determining if the perimeter of the subject has a straight line with a length greater than or equal to a straight-line threshold;
if the perimeter of the subject has a straight line with a length greater than or equal to the straight-line threshold, designating the medical image as having a truncated portion of the subject; and
if the perimeter of the subject does not have a straight line with a length greater than or equal to the straight-line threshold, designating the medical image as not having a truncated portion of the subject.

4. The method of claim 1, wherein determining the medical image has a truncated portion of the subject comprises:
determining a perimeter of the subject in a slice of the medical image;
comparing the perimeter of the subject to an expected perimeter of the subject;
if the perimeter of the subject is outside a tolerance of the expected perimeter of the subject, designating the medical image as having a truncated portion of the subject; and
if the perimeter of the subject is not outside a tolerance of the expected perimeter of the subject, designating the medical image as not having a truncated portion of the subject.

5. The method of claim 4, wherein the expected perimeter of the subject is based on a view of the subject in the slice of the medical image and at least one of gender, age, body mass index, a perimeter measurement, a height measurement, or a measurement of a portion of the subject.

6. The method of claim 1, wherein determining the medical image has a truncated portion of the subject comprises:
presenting the medical image on a display; and
receiving a user input identifying the medical image as having a truncated portion of the subject.

7. The method of claim 1, wherein the non-tissue image values of the truncated portion are identical image values.

8. The method of claim 1, wherein the non-tissue image values of the truncated portion correspond to background image values.

9. The method of claim 1, wherein the medical image comprises a torso of the subject.

10. The method of claim 1, wherein the medical image comprises a lung of the subject.

11. The method of claim 1, wherein the medical image comprises a computed tomography (CT) medical image, a magnetic resonance imaging (MRI) medical image, or a positron emission tomography (PET) medical image.

12. The method of claim 1, further comprising:
defining a region of interest in at least one of the medical image or the reconstructed medical image for application of tumor treating fields to the subject;
creating a three-dimensional model of the subject based on the reconstructed medical image, the three-dimensional model of the subject including the region of interest;
generating a plurality of transducer layouts for application of tumor treating fields to the subject based on the three-dimensional model of the subject;
selecting at least one of the transducer layouts as recommended transducer layouts;
presenting the recommended transducer layouts;
receiving a user selection of at least one recommended transducer layout; and
providing a report for the at least one selected recommended transducer layout.

13. A computer-implemented method for reconstructing a truncated medical image, the method comprising:
accessing from memory a plurality of medical images of a plurality of subjects, the medical images being of a torso of each of the subjects, the medical images comprising voxels;
processing the medical images by truncating a portion of each medical image to obtain truncated medical images, wherein the truncated portion of each medical image corresponds to a right side and a left side of each subject;
designating a set of the truncated medical images as training truncated medical images; and
training a machine learning model to obtain a trained machine learning model, wherein the machine learning model is trained with the training truncated medical images, wherein the machine learning model is trained to generate a reconstructed medical image without truncation.

14. The method of claim 13, further comprising:
designating a set of the truncated medical images as testing truncated medical images, wherein the testing truncated medical images are different than the training truncated medical images;
generating reconstructed medical images using the trained machine learning model and the testing truncated medical images;
comparing the reconstructed medical images to the medical images corresponding to the training truncated medical images to obtain comparison results; and
if the comparison results are unsatisfactory, re-training the trained machine learning model to obtain a re-trained machine learning model.

15. The method of claim 13, wherein the machine learning model is a generative adversarial network.

16. The method of claim 13, wherein the machine learning model is a generative adversarial network comprising a deconvolutional neural network as a generator and a convolutional neural network as a discriminator.

17. The method of claim 13, wherein the trained machine learning model is a deep learning neural network.

18. The method of claim 13, wherein the machine learning model is an unsupervised machine learning model.

19. The method of claim 13, wherein the machine learning model comprises a generative adversarial network (GAN), a MedGAN, a super resolution GAN, a pix2pix GAN, a cycleGAN, a discoGAN, a fila-sGAN, a projective adversarial network (PAN), a variational autoencoder (VAE), or an unsupervised neural network.

20. A computer-implemented method for reconstructing a truncated medical image, the method comprising:
accessing from memory a medical image of a subject, the medical image being of a torso of the subject, the medical image comprising voxels;
determining the medical image has a truncated portion of the subject, the truncated portion of the medical image corresponding to a right side and a left side of the subject, the voxels of the truncated portion having non-tissue image values;
generating, using a trained machine learning model and the medical image, a reconstructed medical image, the trained machine learning model trained to generate a reconstructed medical image without truncation, the voxels of the reconstructed medical image in the truncated portion having tissue image values; and
generating a plurality of transducer layouts for application of tumor treating fields to the subject based on the reconstructed medical image.
